(19) United States
(12) Patent Application Publication
(10) Pub. No.: US 2025/0268731 A1
(43) Pub. Date: Aug. 28, 2025

(54) PROSTHETIC LIMB WITH THERMO-ELECTRIC ACTUATOR

(71) Applicant: Mason Brady, Durango, CO (US)

(72) Inventor: Mason Brady, Durango, CO (US)

(21) Appl. No.: 18/590,653

(22) Filed: Feb. 28, 2024

(51) Int. Cl. A61F 2/58; A61F 2/70

(52) U.S. Cl. CPC A61F 2/586; A61F 2/70; A61F 2002/587; A61F 2002/701

(57) ABSTRACT

A prosthetic apparatus can include: a limb connection portion attachable to a user; a hand portion opposite the limb connection portion, the hand portion including digits; and thermo-electric actuators positioned between the hand portion and the limb connection portion, the thermo-electric actuators connected to the digits.

### FIELD

The described examples relate generally to prosthetic limbs. In particular examples, the disclosure relates to prosthetic limbs having a thermo-electric actuator.

### BACKGROUND

Prosthetics can include functional, artificial replacement limbs for users with amputated, malformed, or missing limbs. Prosthetics can often provide significant transformations to the quality of life for such users. In recent years, prosthetics have undergone great advancements with respect to their functionality and user adaptability. Unfortunately, however, some prosthetics—particularly prosthetic arms—can be too heavy for certain users. Users are often keenly aware of the weight of a prosthetic arm because the center of mass for the prosthetic arm is positioned so far away from the shoulder joint. Adding other components to a prosthetic arm, such as a battery, can add to the weight of the prosthetic arm and exacerbate the perceived effects. Accordingly, at least some conventional prosthetics are too heavy and unwieldy for users, lending to fatigue and lack of fine motor control. Some users can even develop injuries from repetitive compensatory movement patterns due at least in part to the heavy weight of a prosthetic. Therefore, there is a need for an improved prosthetic limb that is lighter and more user friendly.

The subject matter claimed herein is not limited to examples that solve any disadvantages or that operate only in environments such as those described above. Rather, this background is only provided to illustrate one example technology area where some examples described herein may be practiced.

### SUMMARY

An aspect of the present disclosure relates to a prosthetic apparatus that includes: a limb connection portion attachable to a user; a hand portion opposite the limb connection portion, the hand comprising a plurality of digits; and a plurality of thermo-electric actuators positioned between the hand portion and the limb connection portion, the plurality of thermo-electric actuators connected to the plurality of digits.

The prosthetic apparatus can further include a plurality of cables connecting the plurality of thermo-electric actuators to the plurality of digits, wherein upon thermal activation of a thermo-electric actuator of the plurality of thermo-electric actuators, the thermo-electric actuator retracts a cable of the plurality of cables to move a digit of the plurality of digits inward to a retracted position. In some examples, a magnitude of digit retraction to the retracted position is dependent upon a level of the thermal activation. In certain examples, the thermo-electric actuator of the plurality of thermo-electric actuators is configured to transition from an activated state to a resting state by cooling down from a first temperature above an activation temperature to a second temperature below the activation temperature. In at least one example, upon transitioning the thermo-electric actuator of the plurality of thermo-electric actuators from the activated state to the resting state, the digit of the plurality of digits is configured to extend outward and away from the retracted position to a resting position.

The prosthetic apparatus can further include a biasing structure to bias the digit of the plurality of digits toward the resting position. In some examples a first end of the biasing structure is connected to a top portion of the digit between a mid-finger knuckle joint and a fingertip; and a second end of the biasing structure is connected to a top portion of the hand portion between a base knuckle joint and a wrist joint. In one or more examples, each thermo-electric actuator of the plurality of thermo-electric actuators includes at least one Nitinol strand. In particular examples, each thermo-electric actuator of the plurality of thermo-electric actuators includes multiple Nitinol strands, each Nitinol strand within a thermo-electric actuator of the plurality of thermo-electric actuators being independently controllable. In some examples, the prosthetic apparatus can further include a power supply connectable to the plurality of thermo-electric actuators.

Another aspect of the present disclosure relates to a thermo-electric actuator for prosthesis actuation. The thermo-electric actuator can include: a casing defining an internal volume; a first charge plate and a second charge plate positioned within the internal volume; a Nitinol band disposed within the internal volume and having opposing ends electrically coupled to the first charge plate and the second charge plate, respectively; and a movable shaft connected to the first charge plate, the movable shaft attachable to a cable.

In some examples, the Nitinol band has a thermal activation temperature ranging between 30 degrees Celsius and 80 degrees Celsius. In certain examples, the Nitinol band includes a helical coil positionally offset from the casing by a gap; and while maintaining the gap relative to the casing, the helical coil is configured to rotate and compress in response to thermal activation of the Nitinol band. In one or more examples, the casing includes a thermally conductive material configured to dissipate heat from the Nitinol band into an environment external to the casing.

In certain examples, the thermo-electric actuator can include a first insulator insulating the first charge plate from the casing; and a second insulator insulating the second charge plate from the casing. In some examples, the first charge plate and the first insulator define an opening; and the opening is configured to inhibit a pressure differential within the internal volume when the first charge plate and the first insulator is displaced relative to the second charge plate. In one or more examples, the second charge plate is configured to remain stationary within the casing. In particular examples, at least one of the first charge plate or the second charge plate is connectable to a power supply.

Yet another aspect of the present disclosure relates to a thermo-electric actuator for digit actuation of a prosthetic arm. The thermo-electric actuator can include: a casing defining an internal volume, the casing thermally coupling the internal volume to an environment external to the casing; a first charge plate and a second charge plate positioned opposite the first charge plate within the internal volume; a Nitinol helical coil having opposing ends electrically coupled to the first charge plate and the second charge plate, respectively; a movable shaft connected to the first charge plate, the movable shaft attachable to a cable for actuating a prosthetic digit; a seal positioned around the movable shaft; a first insulator insulating the first charge plate from the casing; a second insulator insulating the second charge plate from the casing; and a cap positioned around the second insulator, the second insulator being keyed to the second charge plate and the cap. In some examples, the first charge plate and the first insulator translate together toward the second charge plate when the Nitinol helical coil is heated above an activation temperature.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 illustrates a prosthetic limb in accordance with one or more examples of the present disclosure;

FIGS. 2A-2E illustrate prosthetic digit actuation utilizing a thermo-electric actuator in accordance with one or more examples of the present disclosure;

FIGS. 3-4 illustrate perspective views of a thermo-electric actuator in accordance with one or more examples of the present disclosure;

FIGS. 5A-5B illustrate exploded views of a thermo-electric actuator in accordance with one or more examples of the present disclosure;

FIG. 6 illustrates a profile view of a thermo-electric actuator in accordance with one or more examples of the present disclosure;

FIG. 7 illustrates a cross-sectional view of a thermo-electric actuator in accordance with one or more examples of the present disclosure; and

FIG. 8 illustrates another cross-sectional view of a thermo-electric actuator in accordance with one or more examples of the present disclosure.

### DETAILED DESCRIPTION

Reference will now be made in detail to representative examples illustrated in the accompanying drawings. It should be understood that the following descriptions are not intended to limit the examples to one preferred example. To the contrary, it is intended to cover alternatives, modifications, and equivalents as can be included within the spirit and scope of the described examples as defined by the appended claims.

The following disclosure relates to a lightweight prosthetic apparatus that implements one or more thermo-electric actuators. The prosthetic apparatus of the present disclosure can utilize low levels of energy (e.g., lower energy levels than some conventional prosthetic apparatuses) to efficiently power a thermo-electric actuator, resulting in a smaller and/or lighter power supply. With decreased weight, a prosthetic apparatus of the present disclosure can provide various advantages over conventional prosthetic arms. For example, the disclosed prosthetic apparatus can improve user ergonomics, decrease user fatigue, and increase fine motor control.

The thermo-electric actuator can be implemented in a variety of configurations. In some examples, the thermo-electric actuator includes a linear actuator. In particular examples, the thermo-electric actuator includes a shape memory material (e.g., a nickel titanium alloy known as “Nitinol”) activated by heat. In these or other examples, the thermo-electric actuator can include a pair of charge plates with at least one Nitinol strand electrically coupled to the pair of charge plates. When an electrical charge is selectively applied to the pair of charge plates, the Nitinol strand(s) disposed between the pair of charge plates act as resistance (and therefore build heat) that activates the Nitinol strand(s). Activation of the Nitinol strand(s) can actuate the thermo-electric actuator and a corresponding portion of the prosthetic, such as a prosthetic finger.

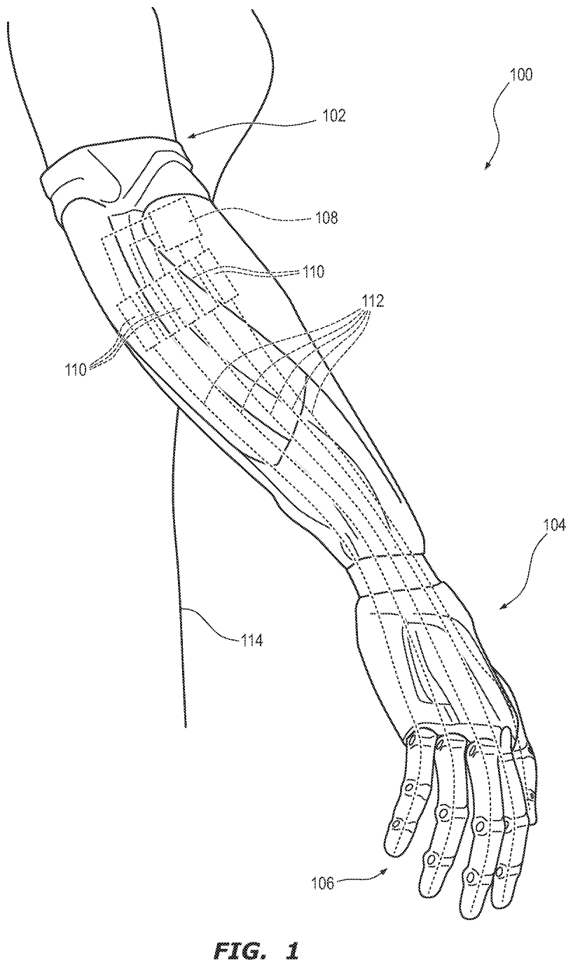

These and other examples are discussed below with reference to FIGS. 1-8. However, a person of ordinary skill in the art will readily appreciate that the detailed description given herein with respect to these figures is for explanatory purposes only and should not be construed as limiting. Furthermore, as used herein, a system, a method, an article, a component, a feature, or a sub-feature including at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates a prosthetic arm 100 in accordance with one or more examples of the present disclosure. As shown, the prosthetic arm 100 can include a limb connection portion 102. The limb connection portion 102 can be sized and shaped to attach to a user 114. For example, the limb connection portion 102 can include a receptacle or socket sized and shaped to receive an end portion of a user's limb (e.g., an amputated limb, malformed limb, or missing limb). In some instances, the socket is a custom socket fabricated to intimately fit, engage, interlock, or mate with the residual limb of the user 114. Additionally or alternatively, the limb connection portion 102 can include a compression sleeve, fastener, belt, sling, harness, etc. to secure the prosthetic arm 100 to the user 114.

The prosthetic arm 100 can further include a hand portion 104 opposite the limb connection portion 102. The hand portion 104 can include digits 106. The digits 106 can include one or more of a finger, hook, grapple, grip, pincer, extension, or other limb-terminating device. In particular examples, the digits 106 can articulate or move (e.g., to bend, touch, pick up an object, squeeze, or point).

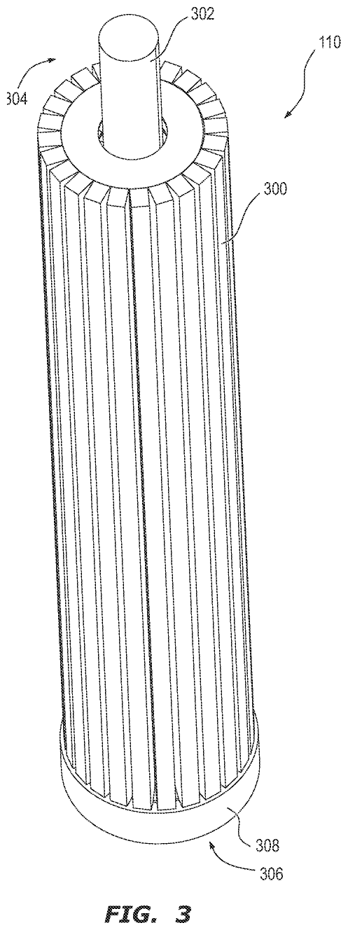

In these or other examples, the digits 106 can articulate in response to actuation of thermo-electric actuators 110. As used herein, the term “thermo-electric actuator” refers to a variety of components, whether mechanical, electromechanical, hydraulic, pneumatic, piezoelectric, thermal, etc., that can generate controlled motion via energy and/or signals (e.g., electrical signals). The controlled motion can, in some examples, include rotary motion or linear motion. In certain examples, a thermo-electric actuator utilizes electrical resistance (particularly the thermal byproduct of resistance) to drive a movable shaft. Additional detail of the thermo-electric actuators 110 is discussed further below in relation to FIGS. 3-8.

The thermo-electric actuators 110 can be positioned at a variety of locations on or within the prosthetic arm 100. In some examples, the thermo-electric actuators 110 are positioned between the hand portion 104 and the limb connection portion 102 (e.g., within the underside of the forearm region). In one or more examples, the thermo-electric actuators 110 are at least partially covered or encapsulated within a body or housing of the prosthetic arm 100 (e.g., to protect the thermo-electric actuators 110 from fluid ingress).

The prosthetic arm 100 can additionally include a power supply 108. As used herein, the term “power supply” refers to any power source that supplies power to one or more components of the prosthetic arm 100 (e.g., to charge a battery and/or power the thermo-electric actuators 110). For example, a power supply can include fuel cells, battery cells, generators, alternators, solar power converters, motion-based converters (e.g., that convert vibrations or oscillations into power), etc. In particular implementations, a power supply can convert alternating current to direct current (or vice-versa) for charging or recharging components of the prosthetic arm 100. Some particular examples of a power supply can include a switched mode power supply, an uninterruptible power supply, an alternating current power supply, a direct current power supply, a regulated power supply, a programmable power supply, a computer power supply, and a linear power supply. In at least one example, a power supply includes a battery (e.g., a rechargeable battery) that can mount onto a battery mount. In a specific implementations, a power supply can include myoelectric technology that uses electrical signals generated by muscles of the user 114 (e.g., in the residual limb) to control movement of the prosthetic arm 100. In one or more examples, the power supply 108 is integrated inside a body or housing of the prosthetic arm 100. In other examples, the power supply 108 is exterior-integrated with the prosthetic arm 100. In these or other examples, the power supply 108 can be electrically coupled with the thermo-electric actuators 110 to selectively actuate the thermo-electric actuators 110.

The thermo-electric actuators 110 can be connected to the digits 106 via cables 112. The cables 112 can include cable ropes, tensioned cable, wire, filament, string, tethers, linkages, pulleys, hinges, plungers, and/or other devices configured to transfer actuation of the thermo-electric actuators 110 to the digits 106. Additionally or alternatively, the cables 112 can include actuatable shafts, toggles, latches, ratchets, clamps, friction brakes, toothed brakes, hydraulic brakes, non-back-drivable mechanisms, bearings/bushings, dovetail slides, scissor mechanisms, sarrus linkages, gears (e.g., worm gears, rotary gears), movable arms, winches, locking mechanisms, etc. In certain examples, the cables 112 can be implemented along a surface of the prosthetic arm 100. In other examples, at least a portion of the cables 112 are positioned sub-surface and/or are at least partially concealed within the prosthetic arm 100.

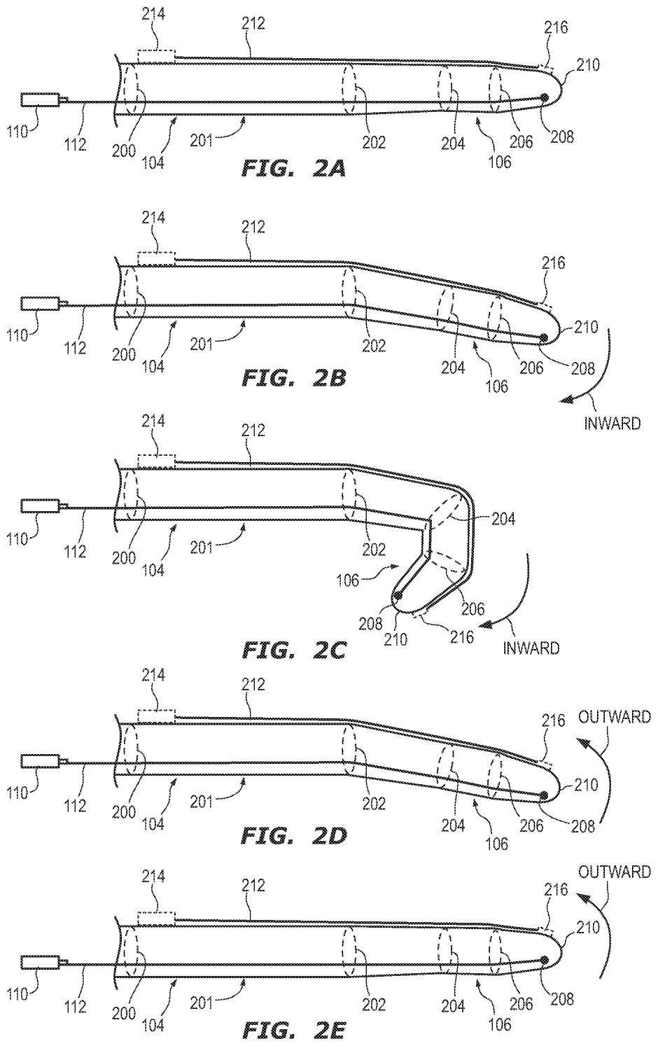

In particular examples, activation of the thermo-electric actuators 110 can cause the cables 112 to retract in and release out for desired digit actuation. For example, each cable of the cables 112 can move a respective digit of the digits 106 in response to actuation of a corresponding thermo-electric actuator 110. For instance, the thermo-electric actuators 110 can retract or tension the cables 112 to cause the digits 106 to correspondingly retract inward (thereby inducing an anatomically-similar flexion of the digits 106 to curve or bend inward toward the palm of the hand portion 104 to a retracted position). Additional detail of example digit actuation or articulation is discussed further below in relation to FIGS. 2A-2E.

It will be appreciated that the prosthetic arm 100 can include a controller to selectively release power from the power supply 108 to the thermo-electric actuators 110. As used herein, the term “controller” can include a processor and a memory device. The processor can include at least one of a system on chip, integrated circuit, driver, microcontroller, application processor, crossover processor, etc. The memory device can include at least one of individual nonvolatile memory, processor-embedded nonvolatile memory, random access memory, memory integrated circuits, DRAM chips, stacked memory modules, storage devices, memory partitions, etc. In these or other examples, the memory device can store computer-executable instructions that, when executed by the processor, cause the processor to perform certain steps, processes, etc. For instance, the processor can cause the power supply 108 to power one or more of the thermo-electric actuators 110. In particular examples, the processor can control an amount and/or duration of current allowed from the power supply 108 to a given thermo-electric actuator (thereby facilitating fine motor control for enabling prolonged object holds, partial depressions or partial finger bends, etc.). That is, the processor can control the level of activation for the thermo-electric actuators 110 (and therefore the different retracted positions of the digits 106) by throttling and/or increasing current flow from the power supply 108 to the thermo-electric actuators 110. In specific implementations, the controller is communicatively coupled to one or more thermistors that are attached to, for example, Nitinol bands of the thermo-electric actuators 110. In some examples, however, the controller is communicatively coupled to the Nitinol bands of the thermo-electric actuators 110 (where the Nitinol bands themselves can serve as thermistors having temperature readouts along one or more portions of the Nitinol bands).

The features of the prosthetic arm 100 can be modified in various ways. For example, although the prosthetic arm 100 is shown as implementing the thermo-electric actuators 110, other types of prosthetic apparatuses can also implement the thermo-electric actuators 110. To illustrate, other prosthetic apparatuses implementing the thermo-electric actuators 110 can include a prosthetic hand, foot, leg, shoulder, etc. As some specific examples, prosthetic apparatus implementing the thermo-electric actuators 110 can include a prosthetic apparatus for partial finger or partial thumb amputations, metacarpal or transmetacarpal amputations, wrist disarticulation, transradial amputation, elbow disarticulation, transhumeral amputation, shoulder disarticulation, forequarter amputation, etc. Accordingly, the prosthetic arm 100 is just one example implementation of a prosthetic apparatus implementing thermo-electric actuators.

As another example modification, the prosthetic arm 100 (or another prosthetic apparatus) can include motor-assist actuation. The motor-assist actuation can work in combination with (or independently from) the thermo-electric actuators 110, as may be desired. Examples of a motor used for such motor-assist actuation can include an alternating current brushless motor, direct current (DC) brushed motor, DC brushless motor, direct drive motor, linear motor, servo motor, stepper motor, etc. Some specific examples of a DC motor include a DC shunt motor, separately excited motor, DC series motor, permanent magnet DC motor, DC compound motor, etc. Some specific examples of an AC motor include a synchronous motor or induction motor. Other examples of an electric motor include a reluctance motor, universal motor, or hysteresis motor.

Any of the features, components, and/or parts, including the arrangements and configurations thereof shown in FIG. 1 can be included, either alone or in any combination, in any of the other examples of devices, features, components, and parts shown in the other figures described herein. Likewise, any of the features, components, and/or parts, including the arrangements and configurations thereof shown and described with reference to the other FIGS. can be included, either alone or in any combination, in the example of the devices, features, components, and parts shown in FIG. 1.

FIGS. 2A-2E illustrate prosthetic digit actuation utilizing a thermo-electric actuator in accordance with one or more examples of the present disclosure. A side profile view of a single digit 106 connected to the hand portion 104 is specifically depicted. As shown in FIG. 2A, the digit 106 is in a rest position (also referred to as an extended position or an unactuated position). In the rest position of FIG. 2A, the digit 106 can be substantially planar or parallel to the hand portion 104. For example, a wrist joint 200, a base knuckle joint 202, a mid-knuckle joint 204, a distal knuckle joint 206, and a fingertip 210 can all be aligned relative to one another.

FIG. 2B shows a first retracted position of the digit 106. The first retracted position of the digit 106 can be achieved by causing the thermo-electric actuator 110 shown in FIG. 2A to actuate (or begin actuating), which in turn tensions or retracts the cable 112. The cable 112 can, in some examples, extend through the wrist joint 200, the base knuckle joint 202, the mid-knuckle joint 204, and the distal knuckle joint 206 before attaching to the fingertip 210 via a connection 208. Thus, the fingertip 210 can begin to draw down and in toward an underside region 201 of the hand portion 104 as the thermo-electric actuators 110 is actuated (e.g., is thermally activated as will be described more below in relation to FIGS. 3-8).

FIG. 2C shows a second retracted position of the digit 106. The second retracted position of the digit 106 can be achieved by causing the thermo-electric actuator 110 shown in FIG. 2B to further actuate, which in turn further tensions or retracts the cable 112. Accordingly, the digit 106 in the second retracted position is—relative to the first retracted position—more curled or drawn inward closer to the palm of the hand portion 104.

Although additional retracted positions are not shown, it will be appreciated that the digit 106 can be incrementally drawn inward to the underside region 201 of the hand portion 104 (e.g., to form a fist, enclose an object, etc.). In other words, the digit 106 can include a continuous range of adjustment resolution that is not bound to certain (e.g., predetermined) intermediate position settings. Of course, the term “continuous” should not be interpreted as necessarily requiring an infinite number of position settings because actuators do not typically have infinite adjustment resolution. Instead, actuators commonly actuate in a pseudo-continuous manner via finely-tuned increments (e.g., increments of micrometers, millimeters, centimeters, inches, etc.). The thermo-electric actuators 110 can also operate in a pseudo-continuous manner such that a magnitude of digit retraction to various retracted positions is dependent upon a level of thermal activation. For example, as the thermo-electric actuators 110 become increasingly thermally activated, the cable 112 can be correspondingly (e.g., proportionally) retracted.

Still, in other examples, the thermo-electric actuators 110 can be actuated in a step-wise fashion or between predetermined positions. For example, the thermo-electric actuators 110 can be actuated between two predetermined positions (e.g., based on Nitinol bands with two specific pre-trained shapes).

After actuated to the desired extent or the desired position, the thermo-electric actuators 110 can transition from an activated state to a resting state. The mechanics of this transition in relation to components of the thermo-electric actuators 110 are discussed further below in conjunction with the description of FIGS. 3-8. In general, however, the thermo-electric actuators 110 can transition from an activated state to a resting state by cooling down from a first temperature above an activation temperature (specific to the thermo-electric actuators 110) to a second temperature below the activation temperature.

FIGS. 2D-2E show this incremental transition of the thermo-electric actuator 110 from an activated state (shown in FIGS. 2B-2C) to a resting state (depicted in FIG. 2E). For example, when the thermo-electric actuator 110 transitions from an activated state to a resting state, the digit 106 can move in an opposite direction (i.e., outward and away from the retracted position of FIG. 2C) to a resting position shown in FIG. 2E (where the digit 106 is substantially relaxed and aligned in-plane with the hand portion 104).

To aid in moving the digit 106 from a retracted position to a resting position, certain embodiments of the prosthetic arm 100 can include a biasing structure 212. The biasing structure 212 can include a spring, elastic band, etc. that can store potential energy as the digits 106 moves inward responsive to actuation of the thermo-electric actuator 110. Then, as the thermo-electric actuator 110 transitions to a resting state, the biasing structure 212 can use the stored potential energy (e.g., in the form of an induced tensile force in the biasing structure 212) to bias or pull the digit 106 back to a resting position. Functionally, in some examples, the biasing structure 212 can provide a spring-back motion for the digit 106 once the thermo-electric actuator 110 is no longer activated. Additionally or alternatively, the biasing structure 212 can help ensure the digit 106 follows a predetermined let-down or path of travel from a retracted position to a resting position.

The biasing structure 212 can be mounted to the prosthetic arm 100 in various locations and orientations. In some examples, the biasing structure 212 is positioned on top of or at least partially embedded within a top portion of the hand portion 104 (e.g., to impart the outward rotational movement for the digit 106). In particular examples, the biasing structure 212 includes an end portion attached to a connection 214 mounted between the base knuckle joint 202 and the wrist joint 200. The biasing structure 212 can include an opposing end portion attached to a connection 216 mounted between the mid-finger knuckle joint 204 and the fingertip 210.

Any of the features, components, and/or parts, including the arrangements and configurations thereof shown in FIGS. 2A-2E can be included, either alone or in any combination, in any of the other examples of devices, features, components, and parts shown in the other figures described herein. Likewise, any of the features, components, and/or parts, including the arrangements and configurations thereof shown and described with reference to the other FIGS. can be included, either alone or in any combination, in the example of the devices, features, components, and parts shown in FIGS. 2A-2E.

FIGS. 3-4 illustrate perspective views of the thermo-electric actuator 110 in accordance with one or more examples of the present disclosure. As shown, the thermo-electric actuator 110 can include a casing 300, a movable shaft 302 extending from a first end 304 opposite a second end 306, and a cap 308.

In more detail, the casing 300 can include a shell, cover, enclosure, frame, sleeve, or collar forming a body portion of the thermo-electric actuator 110. In some examples, the casing 300 thermally couples the internal volume and an environment external to the thermo-electric actuator 110. For instance, the casing 300 can include a thermally conductive material (e.g., aluminum, copper, gold, silver, bismuth, graphene, carbon nanotube, diamond, combinations thereof, etc.) to dissipate heat from components within the internal volume to the environment external to the casing 300. In specific implementations, the casing 300 can also be electrically insulative (e.g., aluminium nitride). For example, the casing 300 can be coated with an electrical insulator, such as a ceramic material.

The casing 300 can also define an internal volume of the thermo-electric actuator 110 where inner components of the thermo-electric actuator 110 can be housed. The casing 300 can therefore include a variety of configurations for an inner housing, capsule, hull, pod, chamber, etc. As shown in other figures, however, the casing 300 can define a cylindrically-shaped internal volume for active displacement of various components of the thermo-electric actuator 110.

As an example, the movable shaft 302 at the first end 304 can move in and out as thermo-electric actuator 110 is actuated (e.g., thermally activated). The movable shaft 302 can extend through a central opening in the casing 300 and attach to a cable (e.g., one of the cables 112 discussed above) for actuating a prosthetic apparatus. Examples of the movable shaft 302 can include a piston, rod, shank, dowel, etc. that moves in tandem with internal components of the thermo-electric actuator 110 (discussed more below). For instance, as the movable shaft 302 retracts inward (i.e., into the casing 300) in response to thermal activation of the thermo-electric actuator 110, the movable shaft 302 can correspondingly pull or tension a cable attached to the movable shaft 302.

With respect to FIG. 4, the thermo-electric actuator 110 can include the cap 308 on the second end 306. In these or other examples, the cap 308 can include a lid, cover, base, or locking mechanism positioned on the second end 306. In some examples, the cap 308 can enclose a bottom end of the casing 300. Additionally or alternatively, the cap 308 can at least partially surround other components of the second end 306. For example, the cap 308 can be positioned around a charge plate 400 and an insulator 402. In some implementations, the cap 308 is integrally formed with the casing 300 at the second end 306. In other implementations, the cap 308 is attached (e.g., adhered or fastened) to the casing 300 at the second end 306.

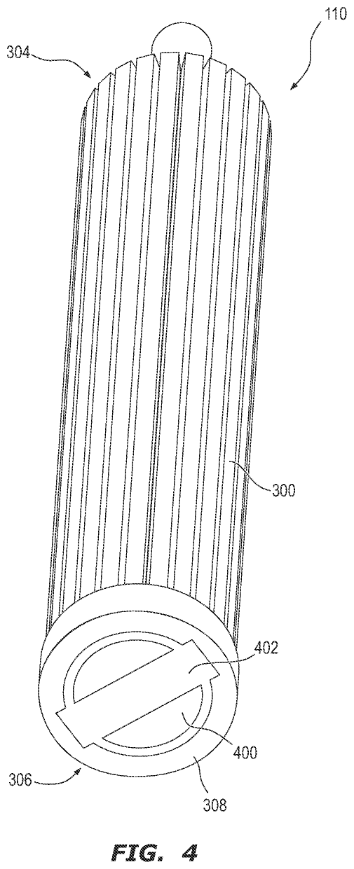

The charge plate 400 can include a stationary base electrically coupled to one or more components within the internal volume of the thermo-electric actuator 110. Additionally or alternatively, the charge plate 400 can be electrically coupled to one or more components external to the thermo-electric actuator 110. For example, the charge plate 400 can be connected to a negative lead on a power supply (e.g., the power supply 108 discussed above). As another example, the charge plate 400 can be connected to an electrical ground (e.g., a chassis or body portion of the prosthetic arm 100). As will be discussed more below, the charge plate 400 can be oppositely charged relative to another charge plate disposed inside the thermo-electric actuator 110 (e.g., the charge plate 502 discussed below in relation to at least FIG. 5). Accordingly, the charge plate 400 can include an electrically conductive material (e.g., copper, aluminum, gold, silver, steel, platinum, zinc, graphite, nickel, brass, tungsten, combinations thereof, etc.).

In one or more examples, the insulator 402 can electrically insulate the charge plate 400 from the casing 300. Electrically insulating the charge plate 400 from the casing 300 can help maintain a desired charge for the charge plate 400 (thereby helping to control thermal activation of the thermo-electric actuator 110). In some examples, the insulator 402 can include a polymer material, composite material, glass material, porcelain material, rubber material, etc.

In specific implementations, one or more components of the second end 306 can interlock with or be keyed to one another. For example, the insulator 402 can be keyed to the charge plate 400 and the cap 308. Additionally or alternatively, the components of the second end 306 can be fastened or bonded together. In this manner, the second end 306 can secure various components of the thermo-electric actuator 110 assembled together and inhibit component creep to maintain relative component spacing.

Any of the features, components, and/or parts, including the arrangements and configurations thereof shown in FIGS. 3-4 can be included, either alone or in any combination, in any of the other examples of devices, features, components, and parts shown in the other figures described herein. Likewise, any of the features, components, and/or parts, including the arrangements and configurations thereof shown and described with reference to the other FIGS. can be included, either alone or in any combination, in the example of the devices, features, components, and parts shown in FIGS. 3-4.

FIGS. 5A-5B illustrates an exploded view of the thermo-electric actuator 110 in accordance with one or more examples of the present disclosure. The thermo-electric actuator 110 can include at least one Nitinol band 500 (or as shown, multiple Nitinol bands 500 spaced apart). The terms “band” and “strand” in conjunction with Nitinol refer to an elongate segment, continuous portion, weaving, filament, fibril, thread, coil, length, mesh, hollow tube, or constituent piece of Nitinol material. A band or strand of Nitinol can have a myriad of different shapes, cross-sectional profiles, and sizes. In some examples, a Nitinol band can include a single filament of Nitinol. In other examples, a Nitinol band can include a combination of multiple Nitinol bands integrally joined (e.g., fused together in a predetermined shape, mesh configuration, tube, linked arrangement, etc.). Still, in other examples, a Nitinol band can include a combination of multiple, discrete Nitinol bands positioned adjacent to each other in intimate contact (e.g., intertwined, twisted, or braided together).

In these or other examples, the Nitinol bands 500 can include various alloys of nickel and titanium. In particular implementations, the Nitinol bands 500 can include a ratio of about 50 atomic percent for each of nickel and titanium (or about 55 percent by weight of nickel). The foregoing ratios, however, can be modified. For instance, the ratio of nickel and titanium can be adjusted to change the austenite yield strength, the activation temperature at which the Nitinol bands 500 can transition to a trained shape (or active state), etc. Additives can also be implemented to tune various properties of the alloy.

In certain examples, the Nitinol bands 500 can include a variety of activation temperatures (e.g., ranging between 30 degrees Celsius and 80 degrees Celsius). The term “activation temperature” refers to the temperature of Nitinol material at which the Nitinol begins to transition between the Martensitic phase and the Austenite phase. As the activation temperature is increased, the time to activation can correspondingly increase (lending to slightly slower response times to retract one or more digits of the prosthetic arm 100). However, with higher activation temperatures, the cooling time to drop below the activation temperate can be correspondingly decreased (lending to slightly faster response times to relax or release one or more digits of the prosthetic arm 100 from a retracted position to a resting position). The converse is also true. As the activation temperature is decreased, the time to activation can correspondingly decrease (lending to slightly faster response times to retract one or more digits of the prosthetic arm 100). Thus, with a lower activation temperature, the cooling time to drop below the activation temperate can be correspondingly increased (lending to slightly slower response times to relax or release one or more digits of the prosthetic arm 100 from a retracted position to a resting position). It will be appreciated, therefore, that the activation temperature of the Nitinol bands 500 can be tuned and adjusted (or customized) for desired digit actuation response rates. In some examples, digit actuation response rates can range from microseconds to seconds.

As mentioned above, the Nitinol bands 500 can include a trained shape such that, upon heating the Nitinol bands 500 above an activation temperature, the Nitinol bands 500 can recover (i.e., move or deform) to the trained shape from another shape (e.g., a non-activated shape, a resting shape, a non-contracted shape, a lengthened shape, a deformed shape, etc.). The trained shape can include any shape, configuration, and/or orientation of the Nitinol bands 500 that occurs above the activation temperature. In specific implementations, the trained shape corresponds to a final shape associated with the Active Austenite Finish (Active Af) temperature of the Nitinol at which the shape recovery is complete. However, it will be appreciated that achieving the trained shape is an incremental process and the Nitinol bands 500 can therefore achieve a portion of the trained shape when a temperature of the Nitinol bands 500 exceeds the activation temperature and progressively warms. The many trainable shapes of the Nitinol bands 500 are herein contemplated. However, in specific implementations of the thermo-electric actuator 110, a trainable shape of the Nitinol bands 500 can include a compressed or contracted helical coil.

Further, in some examples, the Nitinol bands 500 can include multiple trained shapes (e.g., a first trained shape for retracting the thermo-electric actuator 110 and a second trained shape for protracting the thermo-electric actuator 110). In such an example, the biasing structure 212 (or other return mechanism) discussed above may be omitted in lieu of a multiple trainable shapes for the Nitinol bands 500.

In these or other examples, the Nitinol bands 500 can include opposing ends electrically coupled to respective charge plates, namely the charge plate 400 (discussed above) and a charge plate 502 disposed within an internal volume 510 of the thermo-electric actuator 110. The charge plate 502 can be the same as or similar to the charge plate 400. Via the charge plates 400, 502, the Nitinol bands 500 can be thermally activated. For example, the charge plate 502 can be electrically coupled to a positive lead on a power supply (e.g., the power supply 108), and the charge plate 400 can be electrically coupled to a negative lead on a power supply (or an electrical ground). The Nitinol bands 500 can therefore act as resistance in response to an applied or induced charge at the charge plates 400, 502. In turn, the Nitinol bands 500 can be heated to actuate the thermo-electric actuator 110. Specifically, as the Nitinol bands 500 heats above the activation temperature, the Nitinol bands 500 can draw down the charge plate 502 and the movable shaft 302 along axis 508. That is, at least these components can actuate together as a connected system because the Nitinol bands 500 is attached to the charge plate 502, and the charge plate 502 is integrally connected to or otherwise attached to the movable shaft 302.

In some examples, each Nitinol band or strand within the thermo-electric actuator 110 can be independently heat controlled. For example, each end of each Nitinol band can be electrically connected to its own (separate and independent) power connection on the charge plates 400, 502. In this manner, each of the Nitinol bands 500 can be precisely controlled for a more consistent thermal activation (lending to a reduced temperature discrepancy between the Nitinol bands 500 and therefore a more efficient actuating motion for the thermo-electric actuator 110). In these or other examples, each individual band of the bands 500 can have a thermistor connected to the band for precise temperature monitoring and control over the overall actuator contraction rate and the position of the movable shaft 302. Alternatively, in some examples, each individual band of the bands 500 can act as a thermistor itself, having temperature readouts along one or more portions of the Nitinol bands (e.g., for comparing temperatures across different band portions or between different bands).

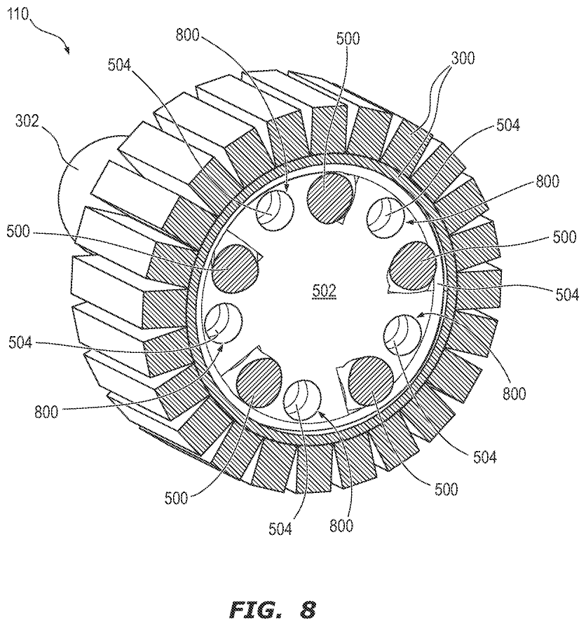

The thermo-electric actuator 110 can further include an insulator 504. The insulator 504 can electrically insulate the charge plate 502 (e.g., from contacting the casing 300). In these or other examples, the insulator 504 can be positioned on top of the charge plate 502 (e.g., to insulate a top surface of the charge plate 502). Additionally, in some examples, the insulator 504 can be positioned around the sides of the charge plate 502 (e.g., to insulate the sidewall of the charge plate 502). In particular implementations, the insulator 504 is adhered, fastened, or otherwise attached to the charge plate 502. The openings of the insulator 504 can correspond to openings in the charge plate 502 (as shown and described in relation to FIG. 8 to inhibit a pressure differential within the internal volume 510 of the thermo-electric actuator 110).

The thermo-electric actuator 110 can additionally include a seal 506 (e.g., a gasket). The seal 506 can be disposed around the movable shaft 302 between the casing 300 and the insulator 504. In some examples, the seal 506 can maintain engagement with the movable shaft 302 as the movable shaft 302 translates in and out of the thermo-electric actuator 110. In so doing, the seal 506 can seal out air, particles, fluids, etc. from being introduced into the internal volume 510.

Any of the features, components, and/or parts, including the arrangements and configurations thereof shown in FIGS. 5A-5B can be included, either alone or in any combination, in any of the other examples of devices, features, components, and parts shown in the other figures described herein. Likewise, any of the features, components, and/or parts, including the arrangements and configurations thereof shown and described with reference to the other FIGS. can be included, either alone or in any combination, in the example of the devices, features, components, and parts shown in FIGS. 5A-5B.

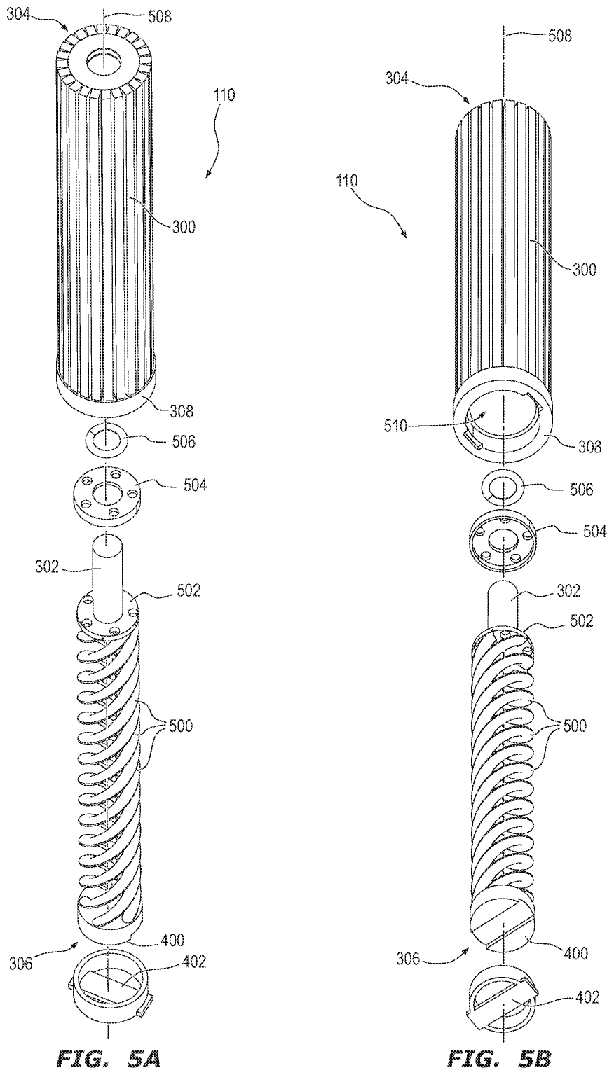

FIG. 6 illustrates a profile view of a thermo-electric actuator in accordance with one or more examples of the present disclosure. In particular, FIG. 6 depicts the thermo-electric actuator 110 as having a length 600. The length 600 can vary depending on the type of prosthetic apparatus or desired actuation. For example, the length 600 can be comparatively larger for actuation of an entire limb (e.g., an arm or leg) than for actuation of digits (e.g., fingers or toes). In particular examples, however, the length 600 can range from about 1 inch to about 8 inches, about 2 inches to about 6 inches, or about 3 inches to about 5 inches.

As mentioned above, the movable shaft 302 can actuate in and out of the thermo-electric actuator 110 to correspondingly draw in and release a cable attached to the movable shaft 302. FIG. 6 specifically depicts an actuation direction 602 for the movable shaft 302. That is, as the Nitinol bands 500 are heated above an activation temperature, the movable shaft 302 is drawn into the thermo-electric actuator 110 along the actuation direction 602.

A displacement distance 604 for the movable shaft 302 can correspond to the magnitude of digit retraction shown in FIGS. 2B-2C. Similarly, the displacement distance 604 can correspond to the level of thermal activation of the Nitinol bands 500. For instance, the displacement distance 604 can increase as the temperature of the Nitinol bands 500 increases beyond the activation temperature and approaches the Active Af temperature of the Nitinol at which the shape recovery is complete.

The displacement distance 604, like the length 600, can depend on the application of the thermo-electric actuator 110. For example, the displacement distance 604 can be larger for actuating an entire limb (e.g., an arm or a leg) versus actuating a digit (e.g., a finger or toe). In particular implementations (e.g., for actuating a digit), the displacement distance 604 can range from about a half centimeter to about 10 centimeters, about 1 centimeter to about 8 centimeters, about 2 centimeters to about 6 centimeters, or about 3 centimeters to about 4 centimeters.

Any of the features, components, and/or parts, including the arrangements and configurations thereof shown in FIG. 6 can be included, either alone or in any combination, in any of the other examples of devices, features, components, and parts shown in the other figures described herein. Likewise, any of the features, components, and/or parts, including the arrangements and configurations thereof shown and described with reference to the other FIGS. can be included, either alone or in any combination, in the example of the devices, features, components, and parts shown in FIG. 6.

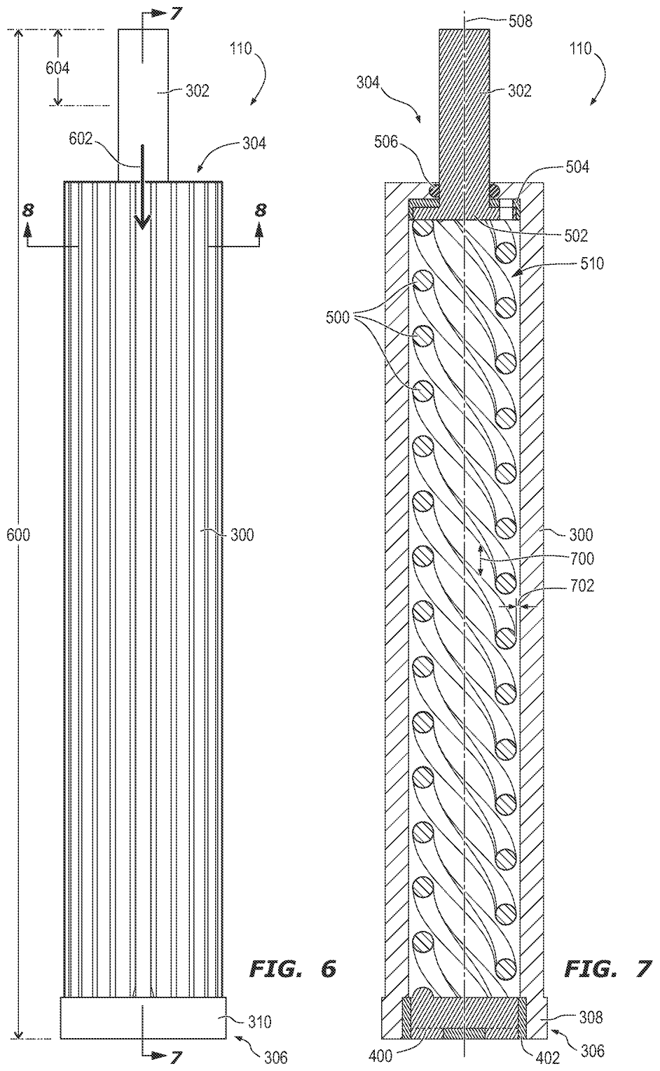

FIGS. 7-8 illustrate cross-sectional views of the thermo-electric actuator 110 in accordance with one or more examples of the present disclosure. In particular, FIG. 7 shows the Nitinol bands 500 disposed within the internal volume 510 and connected to the charge plate 400 and the charge plate 502. As mentioned above, the Nitinol bands 500 can contract in response to an applied charge to the charge plate 400 and the charge plate 502 heating the Nitinol bands 500 above an activation temperature. As the Nitinol bands 500 contracts, each of the charge plate 502, the insulator 504, and the movable shaft 302 can translate together toward the charge plate 400 (i.e., in the actuation direction 602 along the axis 508). As this actuation occurs, the charge plate 400 can remain stationary relative to the casing 300 because the charge plate 400 is attached or adhered to the insulator 402 (which is also stationary and can interlock with or otherwise attach to the cap 308). Additionally, in some examples, the Nitinol bands 500 (and therefore the charge plate 502, the insulator 504, and the movable shaft 302) can rotate about the axis 508 while translating during actuation.

Further, as the Nitinol bands 500 are activated and actuation begins to occur, a spacing 700 between the Nitinol bands 500 can change. For example, the spacing 700 can decrease during as the Nitinol bands 500 are activated and move to a trained shape (e.g., a contracted or compressed helical coil). Conversely, the spacing 700 can increase as the Nitinol bands 500 transition to a resting state and the charge plate 502 translates away from the charge plate 400. In some examples, the spacing 700 is always greater than zero such that the Nitinol bands 500 do not contact each other. In other examples, the spacing 700 can equal zero when the Nitinol bands 500 are activated such that one or more of the Nitinol bands 500 can contact each other (or in the case of a single strand, adjacent coils of the single strand can be compressed into contact with each other).

In these or other examples, the Nitinol bands 500 can maintain a gap 702 relative to the casing 300. In this manner, the Nitinol bands 500 is allowed to flex or move in transitioning to a trained shape, while still avoiding contact with the casing 300. In this manner, the casing 300 is prevented from electrically grounding one or more portions of the Nitinol bands 500. Additionally or alternatively, the gap 702 can help reduce friction (e.g., by preventing friction between the Nitinol bands 500 and the casing 300) to facilitate more efficient actuation of the thermo-electric actuator 110. In some examples, the gap 702 can range from about 0.01 inches to about 0.5 inches, about 0.02 inches to about 0.1 inches, about 0.03 inches to about 0.08 inches, or about 0.05 inches to about 0.07 inches. The gap 702 can also be decreased, for example, due to a coating disposed along the interior wall of the casing 300 (e.g., an electrical insulator coating such as a ceramic coating).

As mentioned above, the casing 300 can dissipate heat from the Nitinol bands 500 into an environment external to the casing 300. In some examples, internal volume 510, can include additional elements to aid heat dissipation. For example, the internal volume 510 can include one or more non-charged fluids to cool the Nitinol bands 500. As some specific examples, a non-charged fluid can include deionized water, oil, air, etc. In such examples, the internal volume 510 can function as a closed-system. In other examples, however, the internal volume 510 can be an open system (e.g., subject to ventilation via fans, pumps, etc. using air from the external environment to actively cool the Nitinol bands 500).

FIG. 8 shows a cross-sectional view of a top portion of the thermo-electric actuator 110 where the Nitinol bands 500 are attached to the charge plate 502. In particular, this figure depicts openings 800 defined by the charge plate 502 and the insulator 504. The openings 800 can inhibit a pressure differential within the internal volume 510 when the charge plate 502 and the insulator 504 are displaced relative to the charge plate 400. In some examples, reducing or eliminating a pressure differential can facilitate a more efficient actuation of the thermo-electric actuator 110 such that displacement of charge plate 502 and the insulator 504 are not hindered.

Any of the features, components, and/or parts, including the arrangements and configurations thereof shown in FIGS. 7-8 can be included, either alone or in any combination, in any of the other examples of devices, features, components, and parts shown in the other figures described herein. Likewise, any of the features, components, and/or parts, including the arrangements and configurations thereof shown and described with reference to the other FIGS. can be included, either alone or in any combination, in the example of the devices, features, components, and parts shown in FIGS. 7-8.

The foregoing description, for purposes of explanation, used specific nomenclature to provide a thorough understanding of the described examples. However, it will be apparent to one skilled in the art that the specific details are not required in order to practice the described examples. Thus, the foregoing descriptions of the specific examples described herein are presented for purposes of illustration and description. They are not intended to be exhaustive or to limit the examples to the precise forms disclosed.

It will be apparent to one of ordinary skill in the art that many modifications and variations are possible in view of the above teachings. Indeed, various inventions have been described herein with reference to certain specific aspects and examples. However, they will be recognized by those skilled in the art that many variations are possible without departing from the scope and spirit of the inventions disclosed herein. Specifically, those inventions set forth in the claims below are intended to cover all variations and modifications of the inventions disclosed without departing from the spirit of the inventions. The terms “including” or “includes” as used in the specification shall have the same meaning as the term “comprising.” The terms “about” or “approximately” should be interpreted as +/−10 percent of a given value, unless otherwise indicated.

What is claimed is:

1. A prosthetic apparatus, comprising:
a limb connection portion attachable to a user;
a hand portion opposite the limb connection portion, the hand portion comprising a plurality of digits; and
a plurality of thermo-electric actuators positioned between the hand portion and the limb connection portion, the plurality of thermo-electric actuators connected to the plurality of digits.

2. The prosthetic apparatus of claim 1, further comprising a plurality of cables connecting the plurality of thermo-electric actuators to the plurality of digits,
wherein upon thermal activation of a thermo-electric actuator of the plurality of thermo-electric actuators, the thermo-electric actuator retracts a cable of the plurality of cables to move a digit of the plurality of digits inward to a retracted position.

3. The prosthetic apparatus of claim 2, wherein a magnitude of digit retraction to the retracted position is dependent upon a level of the thermal activation.

4. The prosthetic apparatus of claim 2, wherein the thermo-electric actuator of the plurality of thermo-electric actuators is configured to transition from an activated state to a resting state by cooling down from a first temperature above an activation temperature to a second temperature below the activation temperature.

5. The prosthetic apparatus of claim 4, wherein upon transitioning the thermo-electric actuator of the plurality of thermo-electric actuators from the activated state to the resting state, the digit of the plurality of digits is configured to extend outward and away from the retracted position to a resting position.

6. The prosthetic apparatus of claim 5, further comprising a biasing structure to bias the digit of the plurality of digits toward the resting position.

7. The prosthetic apparatus of claim 6, wherein:
a first end of the biasing structure is connected to a top portion of the digit between a mid-finger knuckle joint and a fingertip; and
a second end of the biasing structure is connected to a top portion of the hand portion between a base knuckle joint and a wrist joint.

8. The prosthetic apparatus of claim 1, wherein each thermo-electric actuator of the plurality of thermo-electric actuators comprises at least one Nitinol strand.

9. The prosthetic apparatus of claim 8, wherein each thermo-electric actuator of the plurality of thermo-electric actuators comprises multiple Nitinol strands, each Nitinol strand within a thermo-electric actuator of the plurality of thermo-electric actuators being independently controllable.

10. The prosthetic apparatus of claim 1, further comprising a power supply connectable to the plurality of thermo-electric actuators.

11. A thermo-electric actuator for prosthesis actuation, the thermo-electric actuator comprising:
a casing defining an internal volume;
a first charge plate and a second charge plate positioned within the internal volume;
a Nitinol band disposed within the internal volume and having opposing ends electrically coupled to the first charge plate and the second charge plate, respectively; and
a movable shaft connected to the first charge plate, the movable shaft attachable to a cable.

12. The thermo-electric actuator of claim 11, wherein the Nitinol band has a thermal activation temperature ranging between 30 degrees Celsius and 80 degrees Celsius.

13. The thermo-electric actuator of claim 11, wherein:
the Nitinol band comprises a helical coil positionally offset from the casing by a gap; and
while maintaining the gap relative to the casing, the helical coil is configured to rotate and compress in response to thermal activation of the Nitinol band.

14. The thermo-electric actuator of claim 11, wherein the casing comprises a thermally conductive material configured to dissipate heat from the Nitinol band into an environment external to the casing.

15. The thermo-electric actuator of claim 11, further comprising:
a first insulator insulating the first charge plate from the casing; and
a second insulator insulating the second charge plate from the casing.

16. The thermo-electric actuator of claim 15, wherein:
the first charge plate and the first insulator define an opening; and
the opening is configured to inhibit a pressure differential within the internal volume when the first charge plate and the first insulator is displaced relative to the second charge plate.

17. The thermo-electric actuator of claim 11, wherein the second charge plate is configured to remain stationary within the casing.

18. The thermo-electric actuator of claim 11, wherein at least one of the first charge plate or the second charge plate is connectable to a power supply.

19. A thermo-electric actuator for digit actuation of a prosthetic arm, the thermo-electric actuator comprising:
a casing defining an internal volume, the casing thermally coupling the internal volume to an environment external to the casing;
a first charge plate and a second charge plate positioned opposite the first charge plate within the internal volume;
a Nitinol helical coil having opposing ends electrically coupled to the first charge plate and the second charge plate, respectively;
a movable shaft connected to the first charge plate, the movable shaft attachable to a cable for actuating a prosthetic digit;
a seal positioned around the movable shaft;
a first insulator insulating the first charge plate from the casing;
a second insulator insulating the second charge plate from the casing; and
a cap positioned around the second insulator, the second insulator being keyed to the second charge plate and the cap.

20. The thermo-electric actuator of claim 19, wherein the first charge plate and the first insulator translate together toward the second charge plate when the Nitinol helical coil is heated above an activation temperature.
